(19) United States
(12) Patent Application Publication
(10) Pub. No.: US 2015/0031749 A1
(43) Pub. Date: Jan. 29, 2015

(54) BIOMARKER, USES THEREOF AND THERAPY

(71) Applicant: Isis Innovation Limited, Summertown,Oxford (GB)

(72) Inventor: Alison Simmons, Headington (GB)

(73) Assignee: Isis Innovation Limited, Summertown (GB)

(21) Appl. No.: 14/384,647

(22) PCT Filed: Mar. 12, 2013
(86) PCT No.: PCT/GB2013/050602

§ 371 Date: Sep. 11, 2014

(30) Foreign Application Priority Data
Mar. 13, 2012 (GB) 1204391.5

(51) Int. Cl. C12Q 1/68; C12N 15/113

(52) U.S. Cl. CPC C12Q 1/6883; C12N 15/113; C12N 2310/141; C12Q 2600/178; C12N 2320/30
USPC 514/44 A; 435/6.11; 435/6.12; 435/6.19; 506/9; 536/24.33; 536/24.31; 530/389.8; 536/24.5

(57) ABSTRACT

A method of determining the Crohn's disease status of a subject comprising the steps of determining the level of miR-29 in a sample from said subject; and comparing the level of miR-29 determined in step (a) with one or more reference values.

The present invention relates to a novel biomarker and therapy for inflammatory bowel disease, and Crohn's disease in particular, and to uses of the novel biomarker and therapy.

Crohn's disease is a specific inflammatory bowel disease (IBD), the general name for diseases that cause swelling in the intestines. For patients afflicted with Crohn's disease the disease can have a devastating impact on their lifestyle. Common symptoms of Crohn's disease include diarrhoea, cramping, abdominal pain, fever, and even rectal bleeding. Crohn's disease and complications associated with it often result in the patient requiring surgery, often more than once. There is no known cure for Crohn's disease, and long-term, effective treatment options are limited. The goals of treatment are to control inflammation, correct nutritional deficiencies, and relieve symptoms.

Although extensively studied, most of the factors that cause IBD, and Crohn's disease in particular, remain unknown and the current understanding of disease pathogenesis suggests a complex interplay of both environmental and genetic factors.

In spite of considerable research into therapies for IBD, and Crohn's disease in particular, it remains difficult to diagnose. Typically, diagnosis requires a thorough study of the patient's medical history, the exclusion of other conditions, as well as several tests, e.g. blood tests, stool examination, barium enema X-ray, sigmoidoscopy, colonoscopy, and biopsy. Accordingly, there is a need for improved methods for diagnosing Crohn's disease. A simple diagnostic test would therefore be of great benefit both to the patient and the healthcare provider. The present invention fulfils these needs and further provides other related advantages.

In one embodiment the present invention provides means (or markers) which may be used for the diagnosis of Crohn's Disease or a predisposition to Crohn's disease.

In a further embodiment the present invention includes a means for the treatment of inflammatory conditions, and Crohn's disease in particular.

The treatment of patients with Crohn's disease aims to reduce inflammation and promote colon healing and mucosal recovery, and the earlier the disease can be diagnosed, the more likely a treatment is to be successful. Thus improved diagnosis would permit the design of accurate treatment regimes, prevent unnecessary medications and reduce treatment costs.

According to a first aspect, the invention provides a method of determining the Crohn's disease status of a subject comprising the steps of:

(a) determining the level of miR-29 in a sample from said subject; and

(b) comparing the level of miR-29 determined in step (a) with one or more reference values.

miR-29 is a novel biomarker for Crohn's disease.

miR-29, also referred to as microRNA precursor 29 or miRNA-29, is a small non-coding RNA that is involved in regulating gene expression. Reference herein to miR-29 includes miR-29a, miR-29b-1, miR-29b-2 and miR-29c.

miR-29a has the accession number MI0000087, and the sequence:

(Seq ID No: 1)
AUGACUGAUUUCUUUUGGUGUUCAGAGUCAAUAUAAUUUUCUAGCAC
CAUCUGAAAUCGGUUAU

miR-29b-1 has the accession number MI0000105, and the sequence:

(Seq ID No: 2)
CUUCAGGAAGCUGGUUUCAUAUGGUGGUUUAGAUUUAAAUAGUGAUU
GUCUAGCACCAUUUGAAAUCAGUGUUCUUGGGGG

miR-29b-2 has the accession number MI0000107, and the sequence:

(Seq ID No: 3)
CUUCUGGAAGCUGGUUUCACAUGGUGGCUUAGAUUUUUCCAUCUUUG
UAUCUAGCACCAUUUGAAAUCAGUGUUUUAGGAG

miR-29c has the accession number MI0000735, and the sequence:

(Seq ID No: 4)
AUCUCUUACACAGGCUGACCGAUUUCUCCUGGUGUUCAGAGUCUGUU
UUUGUCUAGCACCAUUUGAAAUCGGUUAUGAUGUAGGGGGA

The phrase “Crohn's disease status” includes any distinguishable manifestation of the disease. For example, Crohn's disease status includes, without limitation, the presence or absence of Crohn's disease, the risk of developing Crohn's disease, the stage of Crohn's disease, the progression of Crohn's disease, and the effectiveness or response of a subject to a treatment for Crohn's disease. Reference to Crohn's disease may include Crohn's disease with fibrosis.

The method of the invention may be used, for example, for any one or more of the following: to diagnose Crohn's disease in a subject; to assess the chance of a subject developing Crohn's disease; to advise on the prognosis for a subject with Crohn's disease; to monitor disease progression; and to monitor effectiveness or response of a subject to a treatment.

Preferably the method allows the diagnosis of Crohn's disease in a subject from the analysis of the level of the biomarker in a sample provided by the subject.

The sample material obtained from the subject may comprise peripheral blood cells or myeloid derived cells obtained from a subject. Preferably the sample is not a tissue biopsy, such as a gut biopsy. The sample may be a blood sample, for example, may contain peripheral blood cells or PBMCs. Preferably the sample is assayed for miR-29 levels pre and post pattern recognition receptor triggering of peripheral blood cells.

Preferably determination of the level of miR-29 in a sample comprises the detection of one or more of miR-29a, miR-29b-1, miR-29b-2 and miR-29c, preferably the detection of one or more of Seq ID No: 1, Seq ID No: 2, Seq ID No: 3 and Seq ID No: 4.

The level of the miR-29 present in a sample may be determined by any suitable assay, which may comprise the use of any of the group comprising immunoassays, spectrometry, mass spectrometry, Matrix Assisted Laser Desorption/Ionization Time-of-Flight (MALDI-TOF) Mass Spectrometry, microscopy, northern blot, isoelectric focussing, SDS-PAGE, PCR, quantitative RT-PCR, gel electrophoresis, DNA microarray, and antibody microarray, or combinations thereof. Preferably the level of miR-29 is determined by qPCR.

Preferably the reference value, to which the determined levels of miR-29 are compared, is the level of the miR-29 observed in one or more subjects that do not have any detectable Crohn's disease or any clinical symptoms of Crohn's disease, and have so called “normal values” of the biomarker miR-29.

Preferably miR-29 levels are determined pre and/or post pattern recognition receptor (PRR) triggering. PRR triggering may be achieved by using one or more of MDP, Pam3CSK4, LPS, Flagellin, ssRNA, FSL-1, poly I:C, HKCM and Cpg type A ODN2216.

Preferably if no increase in miR-29 levels is observed in a sample in response to PRR triggering, compared to the level in a normal sample, then this is diagnostic of Crohn's disease.

Alternatively, the reference value may be a previous value obtained for a specific subject. This kind of reference value may be used if the method is to be used to monitor progression of Crohn's disease or to monitor the response of a subject to a particular treatment.

When the determined level of miR-29 is compared with a reference value, an increase or a decrease in the level of the biomarker may be indicative of the Crohn's disease status of the subject.

More specifically a decrease, or no increase, in the level of miR-29 may be indicative, or diagnostic, of Crohn's disease. Preferably the level of miR-29 is determined post PRR triggering.

In this case reference levels may include the initial levels of the biomarker in the subject (for example, before PRR triggering), or the levels of the biomarker in the subject when they were last tested, or both.

Preferably the method of the invention is carried out in vitro.

The subject may be a mammal, and is preferably a human, but may alternatively be a monkey, ape, cat, dog, cow, horse, rabbit or rodent.

The method may further comprise the step of obtaining the sample from the subject.

According to another aspect of the invention there is provided a kit for use in determining the Crohn's disease status of a subject comprising at least one agent for determining the level of miR-29 in a sample provided by the subject.

The agent may be an antibody or a nucleic acid or may be one or more primers for use in a PCR reaction.

The kit may comprise instructions for suitable operational parameters in the form of a label or separate insert. The instructions may inform a consumer about how to collect the sample.

The kit may comprise one or more miRNA samples, to be used as standard(s) for calibration and comparison. The kit may also comprise instructions to compare the level of miR-29 detected in a sample with a calibration sample or chart. The kit may also include instructions indicating what level of miRNA is diagnostic of Crohn's disease.

The kit may also contain agents for use in causing PRR triggering in a sample.

According to a yet further aspect, the invention provides the use of the determination of the level of miR-29 as a means of assessing the Crohn's disease status in an individual.

According to a further aspect the invention provides a primer set comprising two or more primers capable of detecting miR-29. The man skilled in the art will appreciate how to design suitable primers. Such primers may be included in the kit of the invention.

According to another aspect the invention provides a method of treating an inflammatory condition, and Crohn's disease in particular, in a subject comprising administering to the subject an agent capable of modulating the level of miR-29 in a cell. Preferably the miR-29 level is modulated to increase the level of miR-29. The level of miR-29 may be increased by administering miR-29 to the cells, for example, by the use of exosomes. Exosomes may be used to administer miR-29 to dendritic or stromal cells. Alternatively, miR-29 levels may be increased by using an agent, such as an antibody, to prevent the inhibition of the natural expression of miR-29 in cells. Preferably the levels of miR-29 are restored to near or normal levels, or are increased above normal levels. In particular, this method may be used for the treatment of Crohn's with fibrosis.

This method may work to reduce inflammation as the increase in miR-29 levels may act to decrease collagen and hence to decrease inflammation.

According to a still further aspect the method provides an agent for increasing miR-29 levels in a cell, or population of cells, for use in the treatment of an inflammatory condition, and for the treatment of Crohn's disease in particular. The agent may be for use in the treatment of Crohn's with fibrosis. The agent may miR-29, or a mimic of miR-29, or it may be an agent, such as antibody, which acts to prevent the inhibition of miR-29 expression in a cell. The mimic may be a double stranded RNA. Preferably the aim is to restore miR-29 levels to near normal, normal or greater than normal levels.

According to another aspect, the invention provides the use of an agent capable of modulating the level of miR-29 in a cell, for example an agent that increases the miR-29 levels in a cell, in the preparation of a medicament for the treatment of an inflammatory disease, such as Crohn's disease and/or Crohn's disease with fibrosis.

According to another aspect the invention provides the use of an agent capable of modulating the level of miR-29 in a cell, for example an agent that increases the miR-29 levels in a cell, as a therapeutic agent for the treatment of an inflammatory disease, such as Crohn's disease, and Crohn's disease with fibrosis in particular.

The agent may miR-29, or a mimic of miR-29, or it may be an agent, such as antibody, which acts to prevent the inhibition of miR-29 expression in a cell. The mimic may be a double stranded RNA. Preferably the aim is to restore miR-29 levels to near normal, normal or greater than normal levels.

In all therapeutic aspects of the invention it may be intended that miR-29 or a mimic thereof is used as a therapy and is locally administered.

According to a further aspect the invention provides a method of identifying compounds for treating Crohn's disease comprising screening for one or more compounds that increase the level of miR-29 in vitro and/or in vivo.

The invention also provides compounds identified by the above method of identifying compounds.

According to another aspect the invention provides

A system for determining the Crohn's disease status of a subject, said system comprising: a PCR or ELISA assay for determining the level of miR-29 in a sample obtained from the subject; a processor for processing the PCR or ELISA results; computer coded instructions for comparing the results with a database; and a user display for providing the results of the comparison. The database may comprise reference values for miR-29 levels.

The skilled man will appreciate that preferred features of any one embodiment and/or aspect of the invention may be applied to all other embodiments and/or aspects of the invention.

The present invention will be further described in more detail, by way of example only, with reference to the following figures in which:

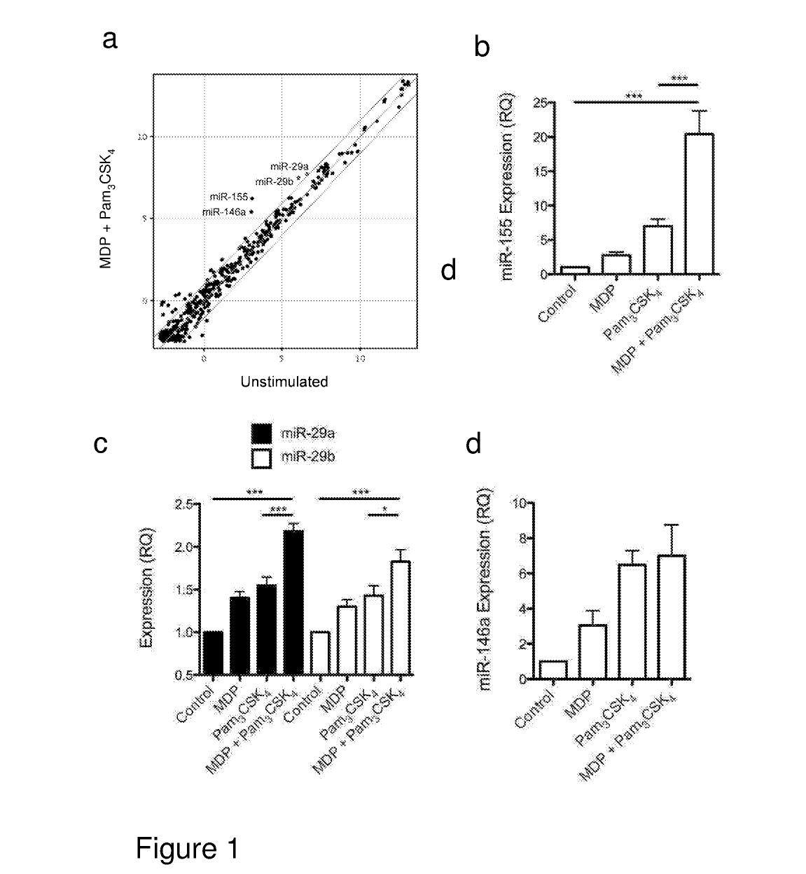

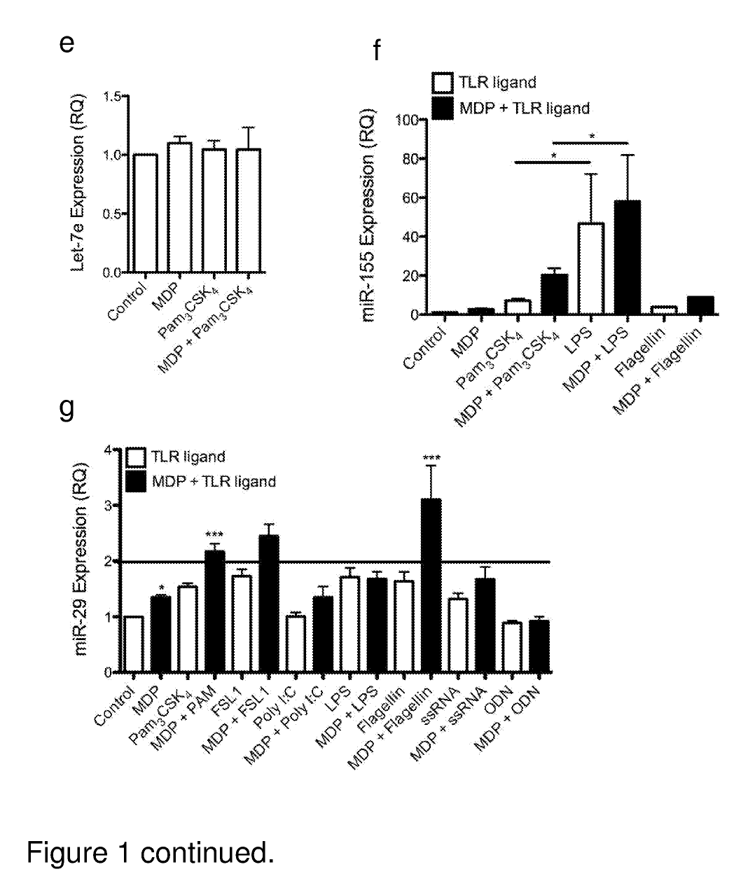

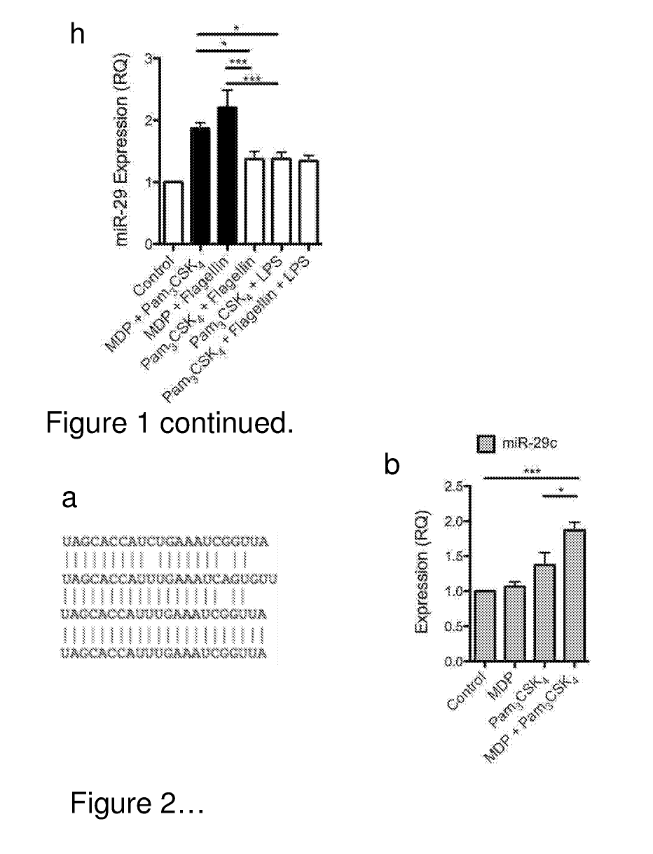

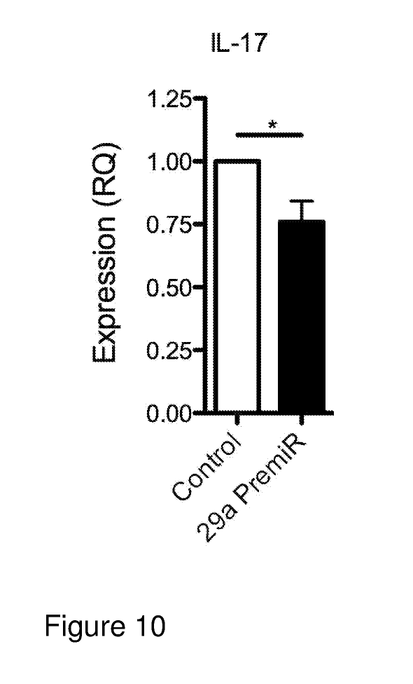

FIG. 1—demonstrates that NOD2 regulates miRNA expression in dendritic cells (DCs). FIG. 1a is a representation of differential regulation of miRNA expression observed by miRNA microarray analysis in DCs stimulated for 24 h with MDP 1 μg/ml and Pam₃CSK₄ 1 μg/ml. The plot shows that miRNAs are more than 2 fold up-regulated by dual stimulation, compared with unstimulated DCs. FIG. 1b shows quantitative real-time PCR (qPCR) analysis of miR-155 expression in DCs stimulated for 24 h with MDP, Pam₃CSK₄ or MDP+Pam₃CSK₄ all at 1 μg/ml, compared with unstimulated cells (control), and relative to non-coding small RNA control RNU44. FIG. 1c shows qPCR analysis as for (FIG. 1b) for miR-29a and miR-29b; miR-146a (FIG. 1d) and miR-Let-7e (FIG. 1e). (FIG. 10 qPCR analysis of miR-155 (FIG. 10 and miR-29 (FIG. 1g) and (FIG. 1h) in DCs stimulated by a panel of PRR ligands alone or in combination as indicated. Ligand concentrations: MDP, Pam₃CSK₄, LPS, Flagellin, ssRNA, FSL-1 at 1 μg/ml; Poly I:C 10 μg/ml; HKLM 10 cells/ml; CpG type A ODN2216 1 μM. Statistical analysis by one-way Anova with Bonferroni post test *P=0.01 to 0.05, **P=0.001 to 0.01, and ***P<0.001. Data are from: 4 biological replicates for array (FIG. 1a), 10 independent experiments (FIG. 1c), 3 independent experiments (FIG. 1b, d, e, f), 16 independent experiments (FIG. 1g), 15 independent experiments (FIG. 1h); error bars, s.e.m.

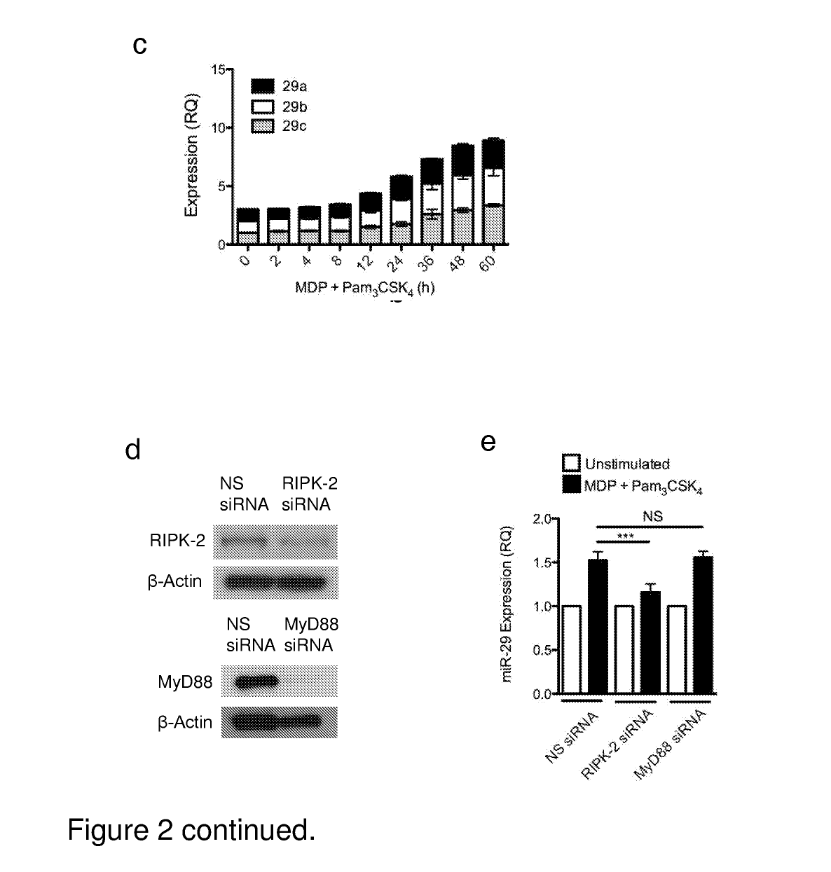

FIG. 2—demonstrates that NOD2 induces miRNA family 29a, 29b, 29c in DCs. FIG. 2a shows a sequence comparison of miR-29a/29b/29c, which form part of two clusters expressed from chromosomes 7 and 1 showing their identical seed sequences. FIG. 2b shows qPCR analysis of miR-29c in DCs stimulated for 24 h with MDP and/or Pam₃CSK₄ (1 μg/ml). FIG. 2c shows qPCR analysis of miR-29a/29b/29c in MDP+Pam₃CSK₄ 1 μg/ml stimulated DCs over time. FIG. 2d shows immunoblot analysis of RIPK-2 and MyD88 expression following transfection of DCs with control nonsense siRNA (NS siRNA) or either RIPK-2 or MyD88 siRNAs. FIG. 2e shows DCs treated as in FIG. 2d after they were left unstimulated or stimulated for 24 h with MDP+Pam₃CSK₄ 1 μg/ml, and miR-29 expression determined by qPCR analysis. Statistical analysis by one-way Anova with Bonferroni post test *P=0.01 to 0.05 and ***P<0.001. Data are from 3 independent experiments (FIG. 2b), 5 independent experiments (FIG. 2c), 6 independent experiments (FIG. 2e); error bars, s.e.m. Western blots are representative of 3 independent experiments (FIG. 2d).

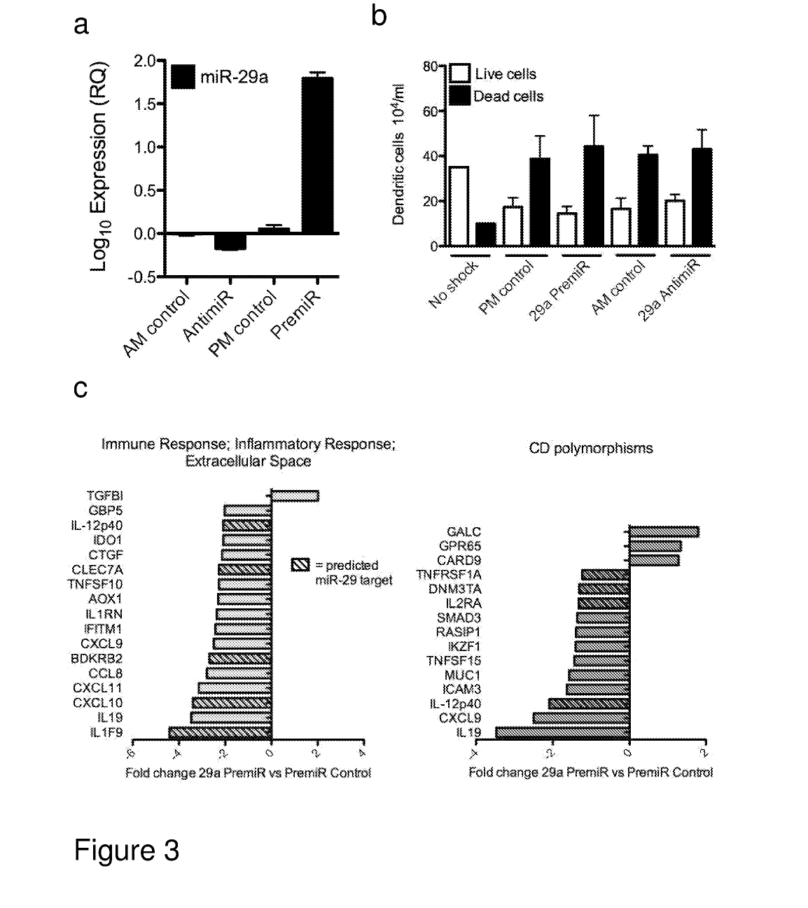

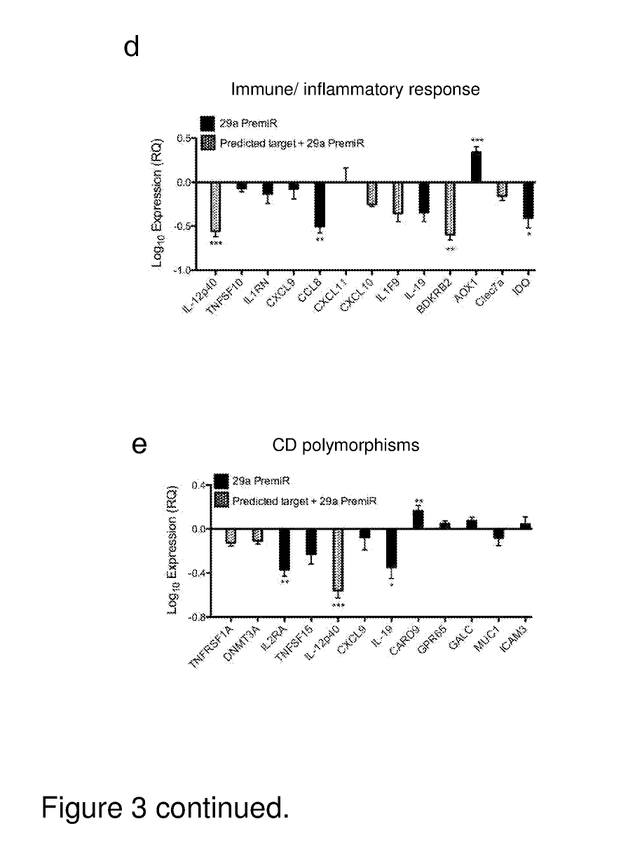

FIG. 3—illustrates that miR-29 regulates immune and inflammatory mediators. FIG. 3a shows qPCR analysis of miR-29a in DCs after transfection of miR-29a antimiR or antimiR control (AM control), or of miR-29a premiR or premiR control (PM control). FIG. 3b shows the live:dead cell ratio, assessed with trypan blue staining, in DCs transfected with miR-29 premiR or antimiR or controls for 24 h before stimulation with MDP+Pam₃CSK₄ 1 μg/ml for 24 h. FIG. 3c shows DCs transfected with miR-29 premiR or PM control for 16 h were then stimulated with MDP+Pam₃CSK₄ whole gene 1 μg/ml for 8 h prior to Agilent human expression microarray analysis. Representation of differential gene expression within immune response; inflammatory response and extracellular space or Crohn's disease (CD) polymorphisms. FIGS. 3d and e shows qPCR analysis of genes identified as differentially expressed by Agilent microarray analysis versus GAPDH control, calculated by the change in threshold (ΔΔC_{T}) method. DCs were transfected for 24 h with miR-29 premiR or PM control, prior to MDP+Pam₃CSK₄ 1 μg/ml stimulation for 24 h. Significant results, by one-way Anova with Bonferroni post test (*P=0.01 to 0.05, **P=0.001 to 0.01, and ***P<0.001). Data from 4 independent experiments (FIGS. 3a and b), from 3 biological replicates for array (FIG. 3c), 4 independent experiments for qPCR (FIG. 3d and e); error bars, s.e.m.

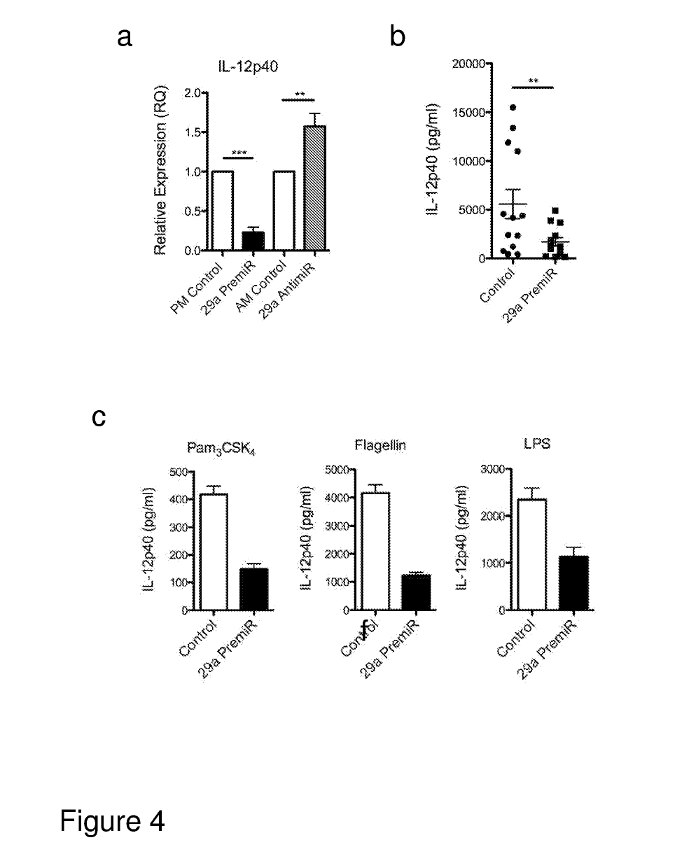

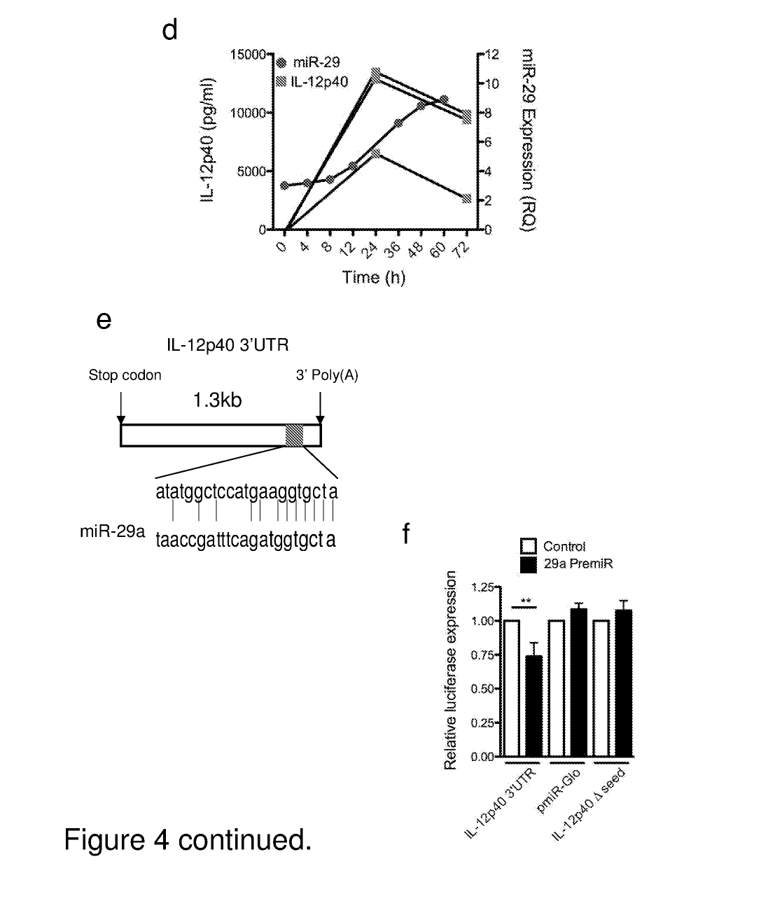

FIG. 4—demonstrates that miR-29 regulates IL-12p40 by directly targeting the 3′UTR. FIG. 4a shows qPCR analysis of IL-12p40 expression in DCs following miR-29 premiR or antimiR or control transfection, and MDP+Pam₃CSK₄ 1 μg/ml stimulation, expressed as relative fold change to control. Statistical analysis by one-way Anova with Bonferroni post test **P=0.001 to 0.01, and ***P<0.001. FIG. 4 shows DCs transfected with miR-29 premiR or control and stimulated with MDP+Pam₃CSK₄ 1 μg/ml for 24 h prior to IL-12p40 ELISA. FIG. 4c shows the results of an IL-12p40 ELISA of DCs treated as in FIG. 4b but stimulated with TLR2/4/5 ligands at 1 μg/ml for 24 h (Analysis by two-tailed paired t-test **P=0.001 to 0.01). FIG. 4d shows the results of an IL-12p40 ELISA (left axis) and miR-29 qPCR (right axis) in DCs stimulated with MDP+Pam₃CSK₄ 1 μg/ml, over time. FIG. 4e shows that the IL-12p40 3′UTR contains a potential target site for miR-29 (TargetScan). FIG. 4f shows the IL-12p40 3′UTR was cloned into pmiR-Glo dual luciferase vector. The vector was co-transfected with miR-29 premiR into HEK293 cells, and firefly luciferase quantified at 48 h (normalised to renilla luciferase activity). The same experiment was performed with the empty vector (pmiR-Glo), or a vector expressing a mutated miR-29 target sequence (IL-12p40seed). Statistical analysis by one-way Anova with Bonferroni post test **P=0.001 to 0.01. Data are from 4 independent experiments (FIG. 4a), 13 independent experiments (FIG. 4b), 2 independent experiments (FIG. 4c), 3 independent experiments (FIG. 4d), 7 independent experiments (FIG. 4f); error bars, s.e.m.

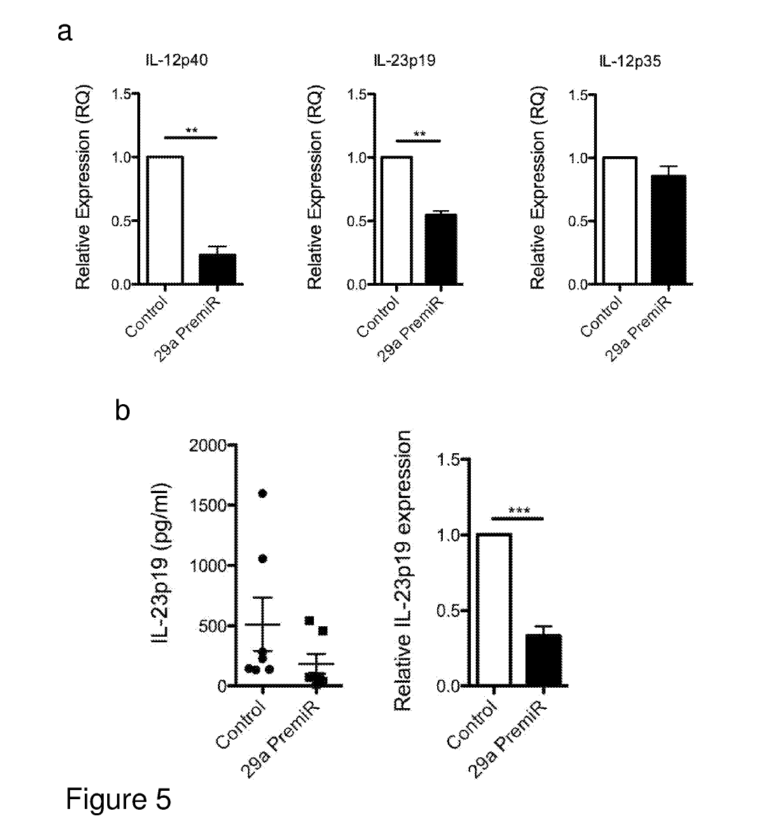

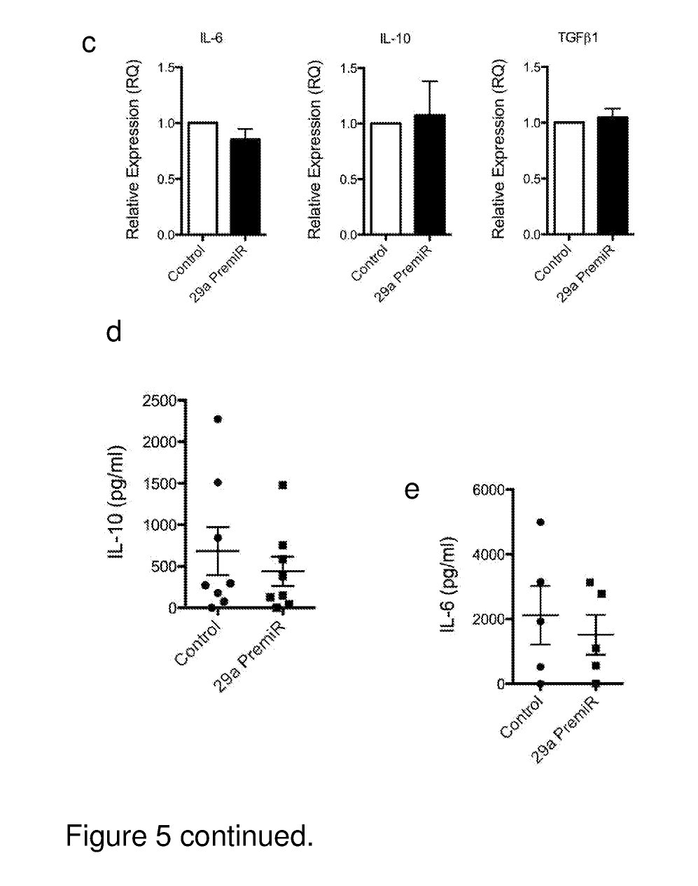

FIG. 5—demonstrates that miR-29 preferentially regulates IL-23. DCs were transfected with miR-29 premiR, or PM control, and stimulated with MDP+Pam₃CSK₄. Graphs analysis of IL-12p40, IL1 μg/ml for 24 h show qPCR-23p19 and IL-12p35 and IL-23p19 ELISA from DCs treated as in FIG. 5a. Significant differences by two-tailed paired t-test **P=0.001 to 0.01 and ***P<0.001. FIG. 5c shows qPCR and FIGS. 5d and e show ELISA (d, e) analysis of DCs transfected with miR-29 premiR, or PM control, and MDP+Pam₃CSK₄ stimulated. IL-1 μg/ml. IL-10 and TGF were assessed by qPCR, and IL-6 and IL-10 by ELISA. Data are from 3 independent experiments (FIG. 5a, FIG. 5d), 7 independent experiments (FIG. 5b), 3 independent experiments (FIG. 5c), 8 independent experiments (FIG. 5d), 5 independent experiments (FIG. 5e); error bars, s.e.m.

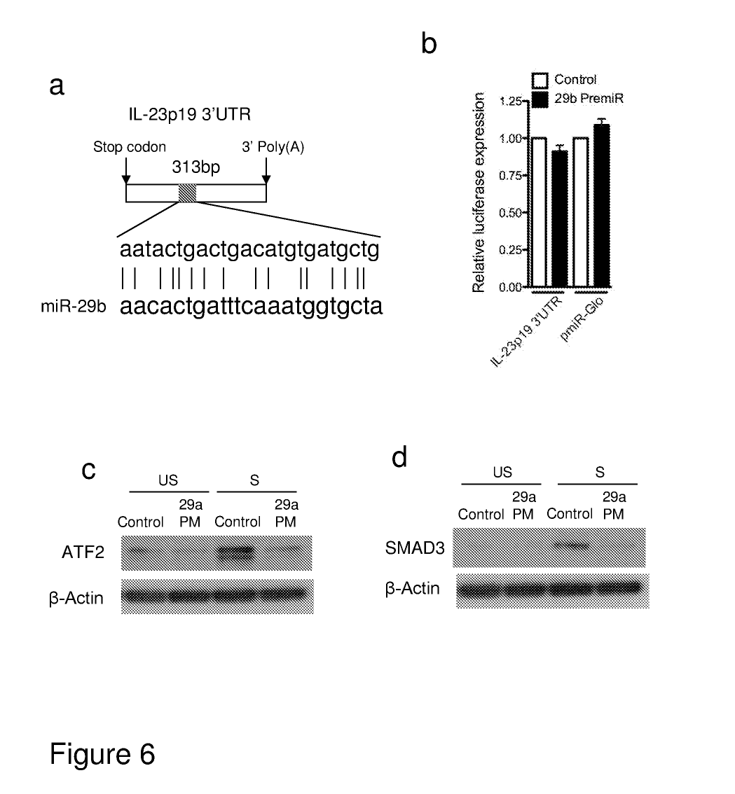

FIG. 6—demonstrates that miR-29 controls IL-23p19 indirectly by regulating ATF2 and SMAD3. FIG. 6a demonstrates that the IL-23p19 3′UTR contains a potential miR-29 target site (MicroCosm). FIG. 6b shows that the IL-23p19 3′ UTR was cloned into pmiR-Glo dual luciferase vector. The vector was co-transfected with miR-29 premiR into HEK293 cells, and firefly luciferase quantified at 48 h (normalised to renilla luciferase activity). FIG. 6c and FIG. 6d show DCs were transfected with miR-29 premiR (29a PM) or control, and subsequently left unstimulated (US) or stimulated with MDP+Pam₃CSK₄ 1 μg/ml (S). Western blot for ATF2 (FIG. 6c) and SMAD3 (FIG. 6d) were performed on the cell lysates. Data are from 3 independent experiments (FIG. 6b), or representative of 3 independent experiments (FIG. 6c, FIG. 6d); error bars, s.e.m.

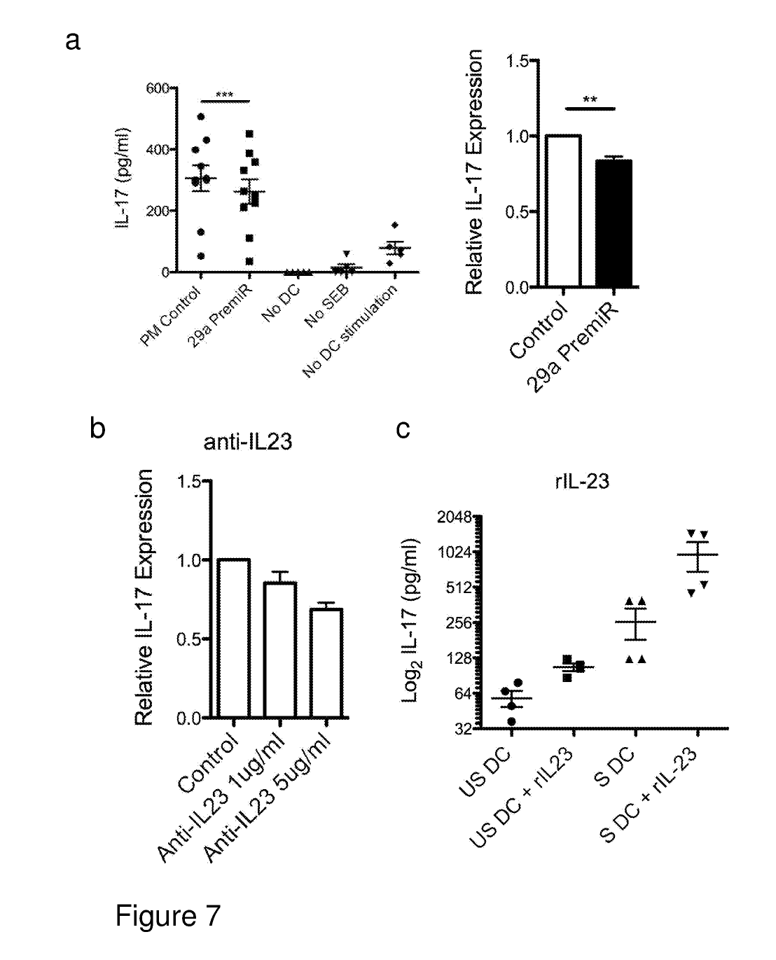

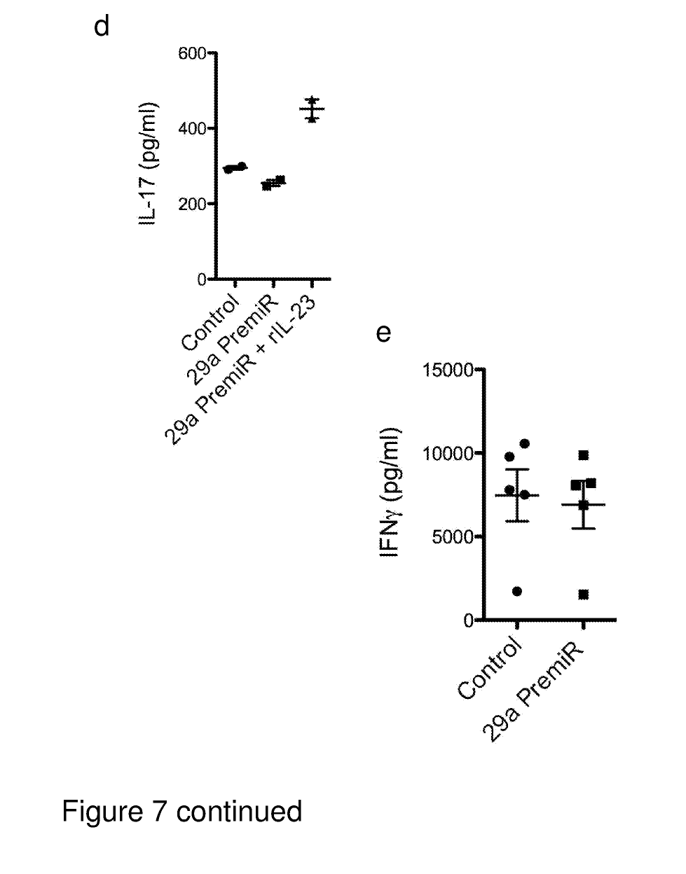

FIG. 7—illustrates the control of IL-17 production from T cells via miR-29. FIG. 7a shows 1×10⁶ CD4+ T cells were co-cultured with 1×10⁵ DCs, with 100 pg/ml SEB. DCs were transfected with miR-29 premiR, or PM control, for 8 h and stimulated with MDP+Pam₃CSK₄ 1 μg/ml for 16 h, before co-culture. IL-17 production after 72 h co-culture was measured by ELISA and qPCR (ELISA shown). Significant differences by two-tailed paired t-test **P=0.001 to 0.01 and ***P<0.001. FIG. 7b shows the results when Anti-IL-23 antibody was added to co-cultures at 1 μg/ml or 5 μg/ml, and IL-17 production assessed at 72 h. FIG. 7c shows the results when recombinant IL-23 (rIL-23) was added at 0.75 ng/ml to co-cultures, and IL-17 production assessed at 72 h. (US=unstimulated DCs; S=MDP+Pam₃CSK₄ stimulated DCs.) FIG. 7d shows the results when DCs were transfected with miR-29 premiR, or PM control, prior to CD4+ T cell co-culture as before. rIL-23 was added at 0.75 ng/ml to co-cultures with DCs expressing the miR-29 premiR. FIG. 7e shows IFN production from DC+T cell co-culture, experimental conditions as for FIG. 7b. Data from 5 independent experiments (FIG. 7a, FIG. 7e), 2 independent experiments (FIG. 7b, FIG. 7d), 4 independent experiments (FIG. 7c); error bars, s.e.m.

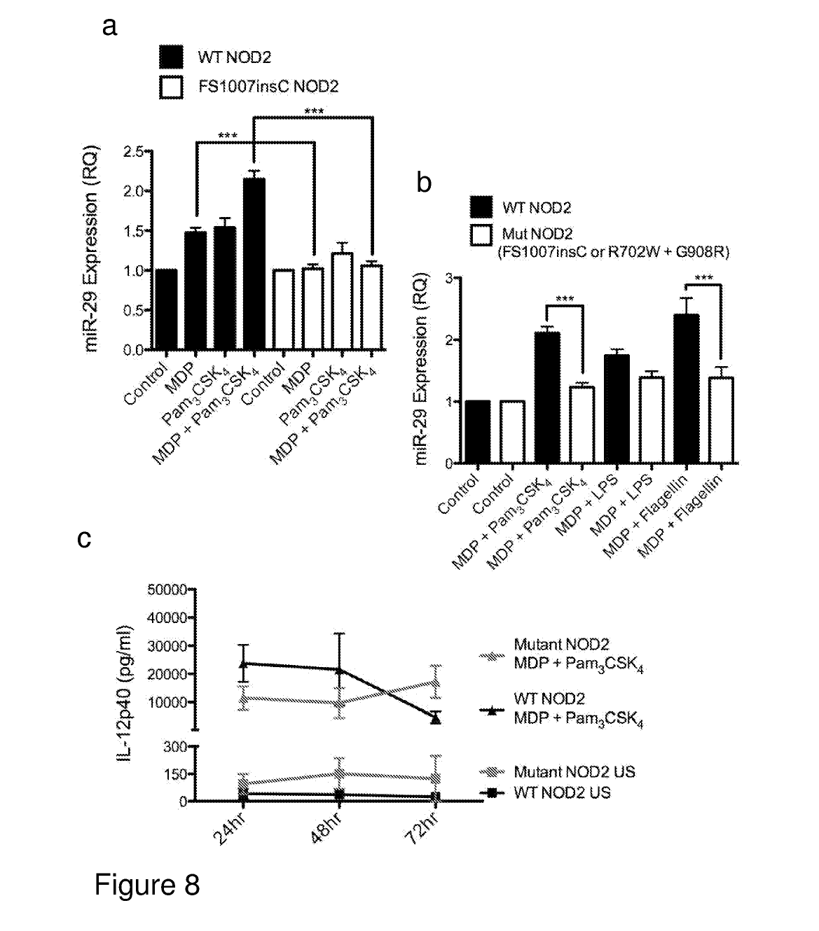

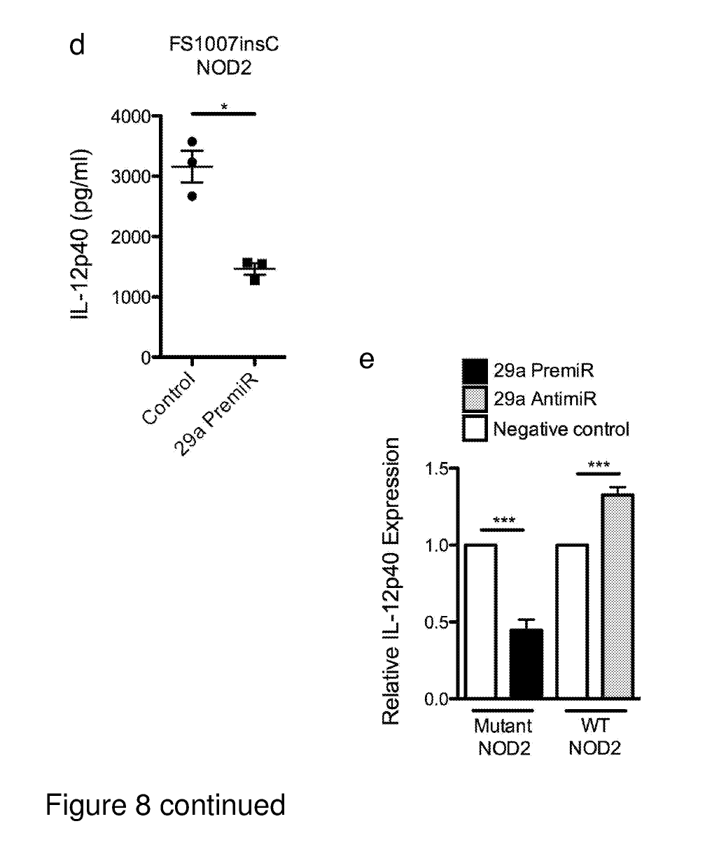

FIG. 8—NOD2 mutant DCs fail to up-regulate miR-29 and control IL-12/23p40. DCs were derived from CD patients homozygous for FS1007insC NOD2 (10 patients), or R702W+G908R NOD2 compound heterozygotes (5 patients). All patients have terminal ileal disease, were in clinical remission and off all immunomodulators/biological therapy. FIG. 8a and FIG. 8b show qPCR analysis of miR-29 expression in DCs expressing or wild-type (WT) NOD2 or Crohn's variant NOD2, after MDP and/or Pam₃CSK₄, or MDP+Flagellin, or MDP+LPS stimulation (1 μg/ml). Statistical analysis by one-way Anova and Bonferroni post test ***P<0.001. FIG. 7c shows IL-12p40 ELISA from WT NOD2, or Crohn's variant NOD2 expressing NOD2 over time. FIG. 8d shows IL-12p40 ELISA following MDP+Pam₃CSK₄ 1 μg/ml stimulation in FS1007insC NOD2 DCs, after transfection of miR-29 premiR or PM control. Significant differences by two-tailed paired t-test (*P=0.01 to 0.05). FIG. 8e shows WT NOD2 DCs transfected with miR-29 antimiR or AM control, and mutant NOD2 DCs transfected with miR-29 premiR, or PM control, before MDP+Pam₃CSK₄ 1 μg/ml stimulation. IL-12p40 expression by ELISA is shown relative to control. Statistical analysis by one-way Anova and Bonferroni post test ***P<0.001. Data are from 8 independent experiments (FIG. 8a), 14 independent experiments (FIG. 8b), 5 independent experiments (FIG. 8c), 3 independent experiments (FIG. 8d, FIG. 8e); error bars, s.e.m.

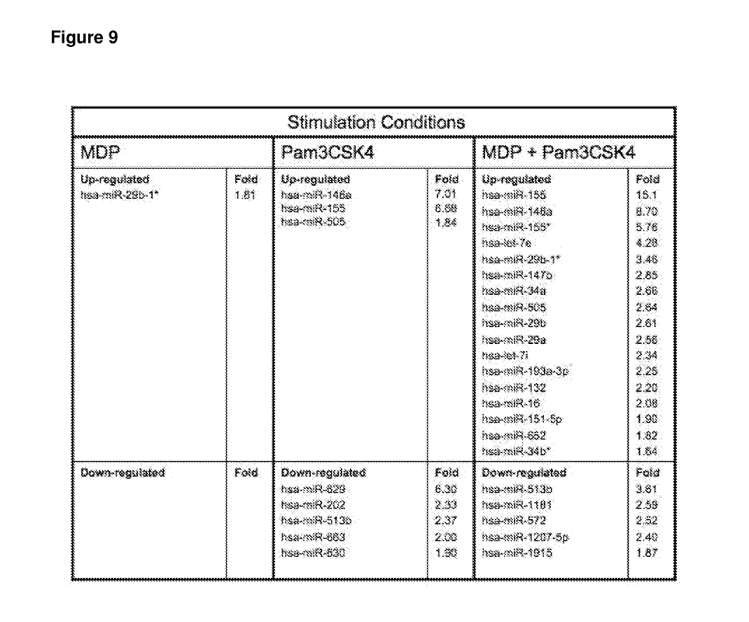

FIG. 9—is a table showing the effect of NOD2 triggering on miR-29 expression.

FIG. 10—demonstrates that there is a significant reduction in IL-17 production from co-cultures where DCs over-express miR-29.

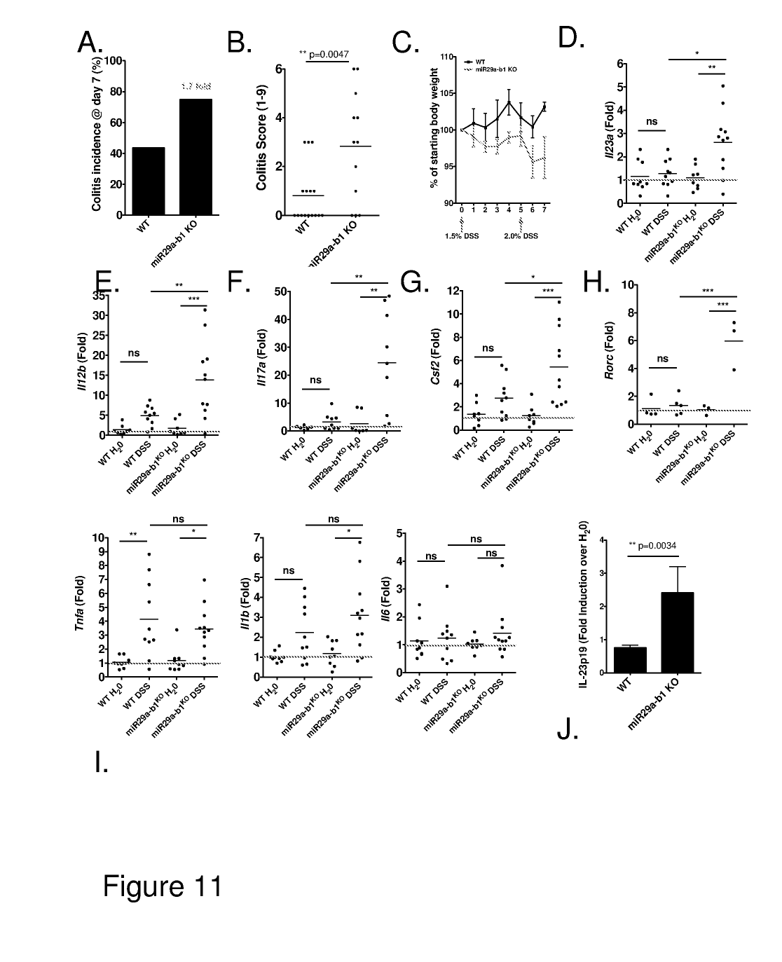

FIG. 11—illustrates that miR29 deficient mice develop exacerbated intestinal inflammation associated with an enhanced Th17 transcriptional signature in colonic tissue. miR29a-b1^{-/-} and WT littermates received low dose DSS in drinking water for 7 days. Weight loss was monitored, and on day 7 mice were sacrificed, colitis scored and distal colonic tissue assessed for mRNA abundance of indicated genes. FIG. 11A and FIG. 11B show colitis incidence and severity of DSS treated WT or miR29-deficient mice FIG. 11C shows weight loss curves. Expression of I123a (FIG. 11D), I112b (FIG. 11E), I117a (FIG. 11F) Csf2 (FIG. 11G) and Rorc (FIG. 11H) mRNA in distal colonic tissue was determined as the fold change in transcript abundance in colitic mice relative to mice of each genotype receiving H₂O. FIG. 11I shows expression levels of the indicated mRNA in distal colonic tissue determined as the fold change in transcript abundance in colitic mice relative to mice of each genotype receiving H₂O. FIG. 11J shows levels of IL-23p19 protein in distal colonic explant cultures from mice of each genotype, expressed as the fold change in levels from colitic mice compared to those receiving H₂O. Data are pooled from 2 independent experiments with n=3-12 mice per group, data in FIG. 11H is from one experiment with n=3-5 mice per group.

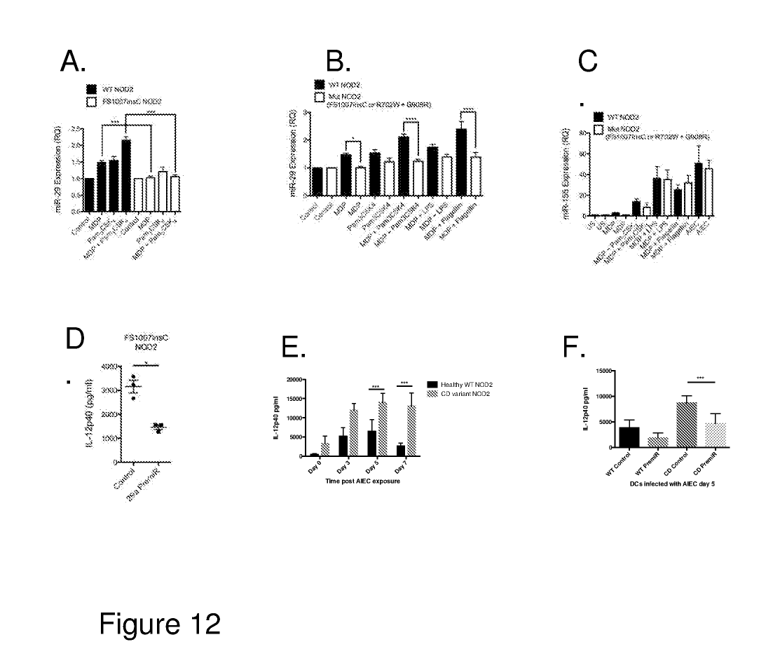

FIG. 12—illustrates Crohn's donor DCs fail to up-regulate miR-29 and control IL-12/23p40. DCs were derived from CD patients homozygous for FS1007insC NOD2 (10 patients), or R702W+G908R NOD2 compound heterozygotes (5 patients). All patients have terminal ileal disease, were in clinical remission and off all immunomodulators/biological therapy. FIG. 12A and FIG. 12B show qPCR analysis of miR-29 expression in DCs expressing or wild-type (WT) NOD2 or Crohn's variant NOD2, after MDP and/or Pam₃CSK₄, or MDP+Flagellin, or MDP+LPS stimulation (1 μg/ml). Statistical analysis by one-way Anova and Bonferroni post test ***P<0.001. FIG. 12C shows qPCR of miR-155 expression in DCs from healthy WT NOD2 expressing DCs vs CD donors expressing NOD2 polymorphisms pre- and post-stimulation with panels of PRR ligands as above. FIG. 12D shows IL-12p40 ELISA following MDP+Pam₃CSK₄ 1 μs/ml stimulation in FS1007insC NOD2 DCs, after transfection of miR-29 premiR or PM control. FIG. 12E shows IL-12p40 release in response to adherent invasive E. coli from healthy donor DCs vs CD DCs at days 0, 3, 5 and 7 post bacterial exposure. FIG. 12F shows IL-12p40 release from healthy donor DCs or CD donor DCs transfected with premiR control or miR-29 premiR for 24 h at day 5 post exposure to adherent invasive E. coli. Statistical analysis by one-way Anova and Bonferroni post test ***P<0.001. Data are from 8 independent experiments A., 14 independent experiments B., 3 independent experiments (C., E.) 2 independent experiments (F); error bars, s.e.m.

### METHODS

### Preparation of Human Monocyte-Derived Dendritic Cells and NOD2 Genotyping

CD14⁺ monocytes were positively selected (anti-CD14 microbeads; Miltenyi Biotech) from peripheral blood mononuclear cells (PBMCs), from either wild-type NOD2 donors, or homozygous mutant NOD2 Crohn's patient donors (Research Ethics Committee Reference: 07/H0603/43). Crohn's disease patients who are homozygous, or compound heterozygous, for NOD2 polymorphisms were identified from the Oxford IBD cohort. All patients in the study have terminal ileal disease (Montreal classification L1 or L3, behaviour B2), and at the time of venesection were in clinical remission; were not on immunomodulators or biological therapy; and were non-smokers. Monocytes were cultured together with IL-4 and GM-CSF (Peprotech). Immature dendritic cells were harvested on day 5 of culture. For NOD2 genotyping PCR of NOD2 polymorphisms (R702W, G908R, FS1007insC) was performed using the primers R702W forward 5′-GAA TTC CTT CAC ATC ACT TTC CAG T-3′ and reverse 5′-GTC AAC TTG AGG TGC CCA ACA TT-3′; G908R forward 5′-CCC AGC TCC TCC CTC TTC-3′ and reverse 5′-AAG TCT GTA ATG TAA AGC CAC-3′; FS1007insC forward 5′-CTG AGC CTT TGT TGA TGA GC-3′ and reverse 5′-TCT TCA ACC ACA TCC CCA TT-3′ prior to sequencing.

Cell Stimulations, miRNA Microarrays and qPCR of miRNAs

5×10⁶ DC's were left unstimulated, or stimulated with 1 μg/ml MDP or 1 μg/ml Pam₃CSK₄ Invivogen), or both, for 24 h. In some experiments a PRR ligand panel was used consisting of lipopolysaccharide (LPS) 1 μg/ml; Poly I:C 10 μg/ml; ssRNA 1 μg/ml; CpG typeA ODN2216 1 μM; HKLM 10⁸ cells/ml; FSL-1 1 μg/ml; and flagellin 1 μg/ml (Invivogen). For miRNA microarrays 4 biological replicates were used. RNA was extracted (miRNeasy; Qiagen), and RNA quality checked using RNA 6000 Nano Assay on Agilent bioanalyzer 2100. Total RNA was hybridized to Agilent human single colour miRNA arrays. Results were analyzed using Genespring. For qPCR of miRNAs RNA was prepared as before. Reverse transcription to cDNA was achieved using miRNA-specific primers (Applied Biosystems) prior to qPCR (TaqMan; Applied Biosystems). Non-coding small RNA control RNU44 (Applied Biosystems) served as an endogenous reference gene, with changes in expression calculated by the change in threshold (CT) method.

miRNA Knockdown or Overexpression and miRNA Target Identification

DCs were nucleofected with miRNA premiR (mimic) or antimiR (miR-29a AM1 2499; miR-29b AM1 0103) final concentration 50 nM, with appropriate negative control (premiR control AM17110; antimiR control AM17010). miRNA mimics are small, chemically modified double-stranded RNAs that mimic endogenous miRNAs and enable miRNA functional analysis by up-regulation of miRNA activity. In this study miRNA mimics were obtained from Dharmacon. Anti-miR inhibitors are short, single stranded 29-O-methyl modified oligonucleotides that are complementary to mature miR-29 sequences and can interact with the miR-RISC complex to inhibit miR function. Potential miR-29 targets were identified using TargetScan, MicroCosm, and MiRanda online algorithms. Agilent dual-color whole human gene expression arrays were employed to identify a further 3×10⁶ targets. DCs were transfected (Lonza, VVPA-1 004) for 16 h with miR-29 premiRs (either miR-29a PM12499, or miR-29b PM1 0103; Applied Biosystems, final concentration 50 nM), or negative control (AM17110, 50 nM). Cells were subsequently stimulated for 8 h with MDP and Pam₃CSK₄ 1 μg/ml. 3 biological replicates were used. Total RNA was extracted (miRNeasy; Qiagen), and RNA quality validated (as per miRNA array), before array hybridization. Genespring was used for data analysis.

### Immunoblots, Antibodies, ELISAs

3×10⁶ DCs were transfected with siRNAs, final concentration 5 nM (RIPK-2 SI02758833; MyD88 SI00300909; NALP1 SI03097619; Qiagen) or non-sense control (AllStar Negative Control, Qiagen) by nucleofection (Lonza). Immunoblot was used to confirm knockdown using anti-human antibodies: anti-RIPK-2, 1:1000 (4982; Cell Signaling); anti-MyD88, 1:1000 (4283; Cell Signaling). For miRNA target confirmation 3×10⁶ DCs were transfected with miR-29 premiR or negative control, as described, and where indicated were stimulated with NOD2+/− TLR ligands. Anti-ATF2, 1:1000 (20F1; Cell Signaling); anti-Smad3, 1:1000 (9523; Cell Signaling) antibodies were used. For ELISAs, DCs were stimulated with NOD2+/− TLR ligands as indicated, +/−transfection of miRNA premiRs or antimiRs with appropriate negative controls. Supernatants were harvested after 48 h unless otherwise indicated, and stored at −80° C. Human IL-12/IL-23p40, IL-6, IL-10, IL-12p70, IL-17 and IFNDuosets, and Human IL-23 Quantikine ELISA Kit, (R&D) were used following standard protocols.

### Luciferase Reporter Assay

Primers for IL-12/IL-23p40 3′ UTR and for IL-23p19 3′ UTR were as follows: IL-12p40 forward 5′-AAA CGA GCT CGC TAG TAG GTT CTG ATC CAG GAT GAA AAT TTG-3′ and reverse 5′-GCA GGT CGA CTC TAG TGA TTA CAA AGA AGA GTT TTT ATT AGT TCA GCC-3′ and for IL-23p19 forward 5′-AAA CGA GCT CGC TAG GGC AGC AGC TCA AGG ATG-3′ and reverse 5′-GCA GGT CGA CTC TAG AGC CAC AAA AAT AAG ACT TTA TTG AA-3′. The 3′UTR was inserted into pmiR-Glo dual-luciferase miRNA target expression vector (Promega), using In-Fusion Dry-Down PCR cloning (Clontech). Mutation of the miRNA seed target sequence in IL-12p40 3′UTR was achieved using QuikChange II XL Site-Directed Mutagenesis Kit (Agilent). Human embryonic kidney (HEK293) cells were co-transfected in 6-well plates with 200 ng vector and 25 nM miRNA premiR or negative control, using lipofectamine 2000 (Invitrogen). Cells were harvested at 48 h before luciferase quantification using Dual-Luciferase reporter assay system (Promega).

### DC+T-cell Co-culture

DCs were prepared as described above. CD4⁺ T-cells were negatively selected from the remaining PBMCs using CD4⁺ T Cell Isolation Kit II (Miltenyi Biotec). DC's were transfected with miRNA premiR or negative control for 8 h before 16 h stimulation with 1 μg/ml MDP and Pam₃CSK₄. 1×10⁵ DCs were co-cultured with 1×10⁶ CD4⁺ T-cells in 12 well plates, with 100 pg/ml SEB. Recombinant IL-23 (1290-IL/CF; R&D, at 0.75 ng/ml or 5 ng/ml) or anti-IL23 antibody (AF1716; R&D, at 1 μg/ml or 5 μg/ml) were added to selected wells. Culture media and cells were collected/harvested for ELISA and qPCR.

### Mice, DSS Challenge, Colitis Scoring and Detection of Inflammatory Mediators in Colonic Tissue

Generation of miR-29a-b1 deficient mice has been described previously (Papadopoulou A S, et al. Nat. Immunol. 13, 181-7. (2011)). Mice were housed under pathogen-free conditions and were used in accordance with the University of Leuven Animal Ethics Committee or UK Scientific Procedures Act 1986. All mice were used at 7-12 weeks of age. For DSS colitis experiments, miR-29a-b1^{-/-} or WT littermate control mice were exposed to 1.5% v/v DSS in their drinking water for 5 days escalating to 2% v/v DSS at day 5. Mice were killed at day 7 and colitis was scored on a severity scale of 1-9, based on percentage weight loss, stool composition and the presence or absence of blood in the intestine. 5 mm² pieces of distal colon were dissected, weighed and cultured in complete medium for 24 hr. Supernatant was collected for IL-23p19p40 quantification by ELISA (R&D Systems, Abingdon, UK), with the concentration of cytokine/ml normalised to total protein mass (in mg) and levels expressed as a fold change in colitic mice over H₂O-treated animals. Frozen pieces of distal colonic tissue were homogenized and RNA extracted using methods as previously described. The relative abundance of target transcripts was determined by qRT-PCR as previously described, using murine Hprt as an endogenous control. Transcript abundance in colitic mice of each genotype was expressed as fold change relative to mice receiving H₂O, using the ddCT method.

Murine DC Stimulations and Quantitation of miR-29 Family Members in Splenic DCs after In Vivo Stimulation

BMDCs from C57BL/6, B6.129, or miR29a-b1^{-/-} mice were obtained from RBC-depleted bone marrow using rGM-CSF (Peprotech, UK) at a concentration of 20 ng/ml in complete RPMI (supplemented with 10% FBS, 100 units/ml penicillin, 100 units/ml streptomycin, 50 μM beta-mercaptoethanol). Cells were cultured at 5% CO₂ and 37° C. in a humidified incubator, with stimulations, transfections and RNA extraction performed as for human cells. mRNA and miRNA transcript abundance was determined by qRT-PCR as previously described, using Hprt and Sno202 as endogenous controls for murine mRNAs and miRNAs, respectively. Murine IL-12p40 levels were determined in BMDC culture supernatant by ELISA (R&D Systems). For in vivo stimulations, 129.RAG^{-/-} and 129.RAG^{-/-}.NOD2^{-/-} mice received PBS or a combination of MDP (100 m/mouse) and PAM₃CSK₄ (5 m/mouse) intravenously. After 24 hours mice were killed and spleens isolated. CD11c⁺ cells were isolated from collagenase-digested tissue using a modified magnetic bead separation protocol (Rehman, A., et al. Gut (2011)). Total RNA was isolated, and miR29 family member abundance was determined by qRT-PCR as described.

### Statistical Analyses

Prism software (GraphPad) was used to determine the statistical significance in the means of experimental groups. When making multiple comparisons on a data set, analysis was by one-way Anova with Bonferroni post test. For experiments with two sample groups (one condition, one control) and a single comparison, analysis was by paired, two-tailed Student's t-test.

### Background

40% of Western Crohn's disease (CD) patients carry one of three mutations in the NOD2 gene, namely amino-acid substitutions Arg702Trp and Gly908Arg and the frameshift FS1007insC, all of which are found within a leucine rich repeat region which is responsible for muramyl dipeptide (MDP) recognition.

NOD2 is a cytosolic pattern recognition receptor (PRR) that controls immunity against intracellular bacteria and inflammatory responses. NOD2 recognizes MDP, an integral component of bacterial cell walls, and is expressed exclusively in monocyte lineage cells, intestinal epithelial cells and Paneth cells.

The functional role of NOD2 is not completely defined, in particular the mechanism by which it signals in dendritic cells (DCs) is unclear. Like other PRRs, it can induce NF-kB activation but in comparison with PRRs such as the Toll-like receptors (TLRs) this effect is rather weak. Large-scale gene expression studies have shown NOD2 can synergize with other PRRs in differential gene regulation and that this synergy is lost in cells expressing Crohn's disease variant NOD2. NOD2 plays a key role in amplifying release of certain pro-inflammatory cytokines, particularly IL-6 and IL-23 from DCs and macrophages. IL-6 and IL-23 are required for induction of Th17 CD4⁺ T cells, a response important for anti-microbial immunity at mucosal surfaces and a hallmark of the inflammatory response in Crohn's disease. The significance of the IL-23/Th17 pathway for Crohn's disease pathogenesis was highlighted by genetic studies, with polymorphisms in IL23R, IL12B (IL-12p40), STAT3, JAK2 and TYK2 all contributing to disease predisposition.

The results presented herein demonstrate that NOD2 can regulate miRNA expression in DCs. Of particular interest, NOD2 is required for induction of the miR-29, including miR-29a, miR-29b-1, miR29b-2 and miR-29c. Overexpression of miR-29 together with large scale gene expression profiling allowed a number of miR-29 target genes within inflammatory/immune pathways to be identified, including genes not previously described as containing Crohn's disease susceptibility polymorphisms. The results demonstrate that miR-29 directly targets IL-12p40 to down-regulate release of IL-12p40 from DCs, and targets the IL-23p19 transcriptional activators ATF2 and SMAD3 to repress IL-23p19 expression. miR-29 overexpression down-regulates IL-23 release from DCs and attenuates Th17 CD4+ T cell responses. Crohn's disease DCs expressing associated NOD2 variants are shown to be incapable of inducing miR-29 following NOD2 triggering. The data further demonstrates that during combinatorial PRR triggering patient's cells show reduced ability to return this pro-inflammatory cytokine production to basal levels; an effect that is reversible on overexpression of a miR-29 premiR (mimic). Thus miR-29 induction mediated through NOD2 is pivotal for down-regulation of IL-23 at the end of an immune response, and loss of this effect may contribute to abnormal elevation of IL-23 observed in Crohn's disease. miR-29 is also shown to downregulate IL-12p40/IL-23 expression in murine DCs and miR-29a knockout (KO) mice show worsened colitis on DSS challenge, together with raised IL-23 levels and Th17 signature genes in the intestinal mucosa. The results demonstrate that the decrease in miR-29 levels may be used as a diagnostic tool for use in the diagnosis of Crohn's disease, and that modulation of miR-29 levels may be used for therapeutic purposes.

### Results

NOD2 Affects miRNA Expression in DCs

To demonstrate that NOD2 triggering by MDP could induce differential miRNA expression in DCs, immature monocyte-derived DCs expressing wild type (WT) NOD2 with MDP were stimulated and miRNA expression was measured by miRNA microarray analysis at 24 h. The miRNA microarrays used were from Agilent and represented 866 human and 89 human viral miRNAs sourced from Sanger miRBase (release 1 2.0). On NOD2 triggering alone very little differential regulation of miRNAs was observed; only miR-29 was induced (FIG. 9). By comparison, stimulation of TLR2 with Pam₃CSK₄ led to robust differential regulation of miRNAs with strong induction of miR-155 and miR-146 previously described as being induced on TLR triggering and DC maturation (FIG. 9). NOD2 has previously been shown to cross talk with TLR2, and as both these PRRs recognize different components of peptidoglycan it is likely they would normally be co-triggered on bacterial recognition. The results of single triggering of either NOD2 or TLR2 were compared with that occurring on dual stimulation with MDP and Pam₃CSK₄. This led to increased (synergistic) differential regulation of a number of miRNA as regulated by TLR2 alone and in addition greatly increased induction of miR-29 induced by NOD2 (FIG. 1a). To confirm the accuracy of the results obtained by microarray analysis, quantitative PCR (qPCR) of the most strongly induced miRNAs was undertaken on NOD2+ TLR2 co-triggering and this confirmed a strong synergy between NOD2 and TLR2 in up-regulation of miR-155 (FIG. 1b) and miR-29a/miR-29b (FIG. 1c), but not miR-146a (where NOD2 stimulation contributed little to overall induction via TLR2) (FIG. 1d) or miR-Let-7e (where little induction occurred across all stimuli used) (FIG. 1e). miR-155 is the most well characterized miRNA utilised by the immune system and it is well known that it is induced on PRR signaling. A panel of PRR ligands were used to compare the effect of NOD2 on miR-155 induction in comparison with other PRRs. Activation of TLR4 by lipopolysaccharide (LPS) dwarfed the effect of NOD2+ TLR2 triggering on miR-155 expression, suggesting NOD2 is unlikely to play a major role in miR-155 in physiological circumstances when DCs encounter microbes (FIG. 10. In contrast NOD2 played a dominant role in the induction of miR-29, where greatest up regulation was observed with either NOD2+ TLR2 triggering or NOD2+ TLR5 triggering (FIG. 1g). NOD2 was shown to be required for this effect as combined TLR2+ TLR5 triggering did not result in miR-29 up-regulation (FIG. 1h). This data revealed an exclusive role for NOD2, in comparison with other PRRs, in inducing miR-29 in DCs.

NOD2 Induces miRNA Family 29a, 29b, 29c in DCs

miR-29 forms part of an miRNA family expressed from two clusters on chromosomes 1 and 7, and possessing identical seed sequences, therefore targeting the same endogenous mRNAs (FIG. 2a). The data presented herein demonstrates that, in addition to miR-29a and miR-29b, miR-29c is also induced on NOD2 or NOD2+ TLR2 dual triggering. The level of induction of miR-29c was observed to be similar to miR-29a or miR-29b following combined NOD2+ TLR2 stimulation (FIG. 2b). Induction of the miR-29 family increased over time after NOD2+ TLR2 dual stimulation, being detectable from 8 h post-stimulation and peaking around day 3 (FIG. 2c).

miR-29 Targets Multiple Inflammatory Genes in DCs Including IL-12p40

Computational methods (including TargetScan, and miRanda algorithms) and functional biological experiments were used to seek targets of miR-29. Previously it has been shown that miRNAs mostly act to down-regulate mRNA of target genes, rather than at the translational level in mammalian cells. NOD2+ TLR2 stimulated DCs were transfected with miR-29 premiR to artificially increase miR-29 expression and determine, by gene expression microarray analysis, potential target genes among mRNAs that were differentially regulated. Transfection of miR-29 premiR resulted in robust up-regulation of miR-29 sequence detected by qPCR (FIG. 3a). As miR-29 had previously been shown to affect expression of genes controlling cell death such as MCL-124, studies were undertaken to determine whether induction of miR-29 levels by overexpression of miR-29 premiR, or blocking miR-29 action with an antimiR (antagomir), changed the rate of cell death. No significant change in dead cells by Trypan blue staining between cells transfected with miR-29 premiR, or miR-29 antimiR, was observed over the time course of the experiment (FIG. 3b). Large scale gene expression profiling was undertaken of NOD2+ TLR2 stimulated DCs transfected with either miR-29 premiR or control. Amongst the differentially regulated mRNAs a bias was observed in genes previously described as functioning in immune or inflammatory pathways or as IBD susceptibility genes (FIG. 3c). A number of the putative targets differentially regulated by miR-29 were confirmed by qPCR (FIGS. 3d and 3e). One of the most strongly down-regulated genes identified by this methodology was IL-12p40, a subunit of IL-12 and IL-23 and a known IBD susceptibility gene. Thus miR-29 exerts broad control over a number of inflammatory and immune pathway genes including known IBD susceptibility genes such as IL-12p40.

miR-29 Down-Regulates and Directly Targets IL-12p40

As IL-12p40 was the most strongly down-regulated gene observed following overexpression of miR-29 premiR, and as it is predicted to be directly targeted by miR-29 by computation algorithms, the role of miR-29 in the control of IL-12p40 expression was studied further. The effect of miR-29 on IL-12p40 expression was studied by transfecting miR-29 premiR and performing qPCR for IL-12p40, or ELISA for IL-12p40 release, from DCs after NOD2+ TLR2 stimulation. Overexpression of miR-29 led to marked down-regulation of both IL-12p40 mRNA levels and cytokine release from DCs (FIGS. 4a and 4b), and this was irrespective of the PRR stimulus used to induce IL-12p40 (FIG. 4c). Moreover, blocking the effect of miR-29 with an antimiR, in MDP+Pam₃CSK₄ treated DCs increased IL-12p40 expression (FIG. 4a). The kinetics of IL-12p40 down-regulation over time post NOD2+ TLR2 triggering inversely correlates with the kinetics of miR-29 induction, with the most exaggerated effect observed at 3 days, consistent with miR-29 acting to repress IL-12p40 physiologically (FIG. 4d). miR-29 is predicted to directly target the IL-12p40 3′ UTR (FIG. 4e). To study this the 3′ UTR (>3,000 nucleotides) of IL-12p40 mRNA was cloned into a vector downstream of a reporter gene encoding luciferase. HEK293 cells were then transfected with this vector, or control empty vector, along with miR-29 premiR for 48 h. Reporter gene expression was suppressed by 25% in cells carrying the vector containing the predicted binding site for the miR-29 family (FIG. 4f). These results were confirmed by mutating the IL-12p40 3′ UTR seed sequence and observing that suppression of reporter gene expression was reversed in the presence of the mutant 3′ UTR. These experiments confirm IL-12p40 is a direct target of miR-29 in DCs, and show an inverse correlation between expression of miR-29 and IL-12p40 induction over time post-PRR stimulation. This regulation of IL-12p40 expression is consistent with miR-29 functioning as a late induced miRNA, acting to switch off expression of this inflammatory mediator.

miR-29 Down-Regulates IL-23

IL-12p40 is both a subunit of IL-12 and IL-23, the other subunits being IL-12p35 and IL-23p19 respectively. Studies were undertaken to determine whether miR-29 controlled expression of either of these other two subunits that act in concert with IL-12p40, these studies involved transfecting DCs with miR-29 premiR or a control and performing qPCR to study the expression of IL-12p35 and IL-23p19. FIG. 5a shows that miR-29 premiR strongly down-regulates expression of IL-12p40 and IL-23p19, but not IL-12p35. miR-29 also robustly down-regulates IL-23p19 protein released from DCs on NOD2+ TLR2 triggering (FIG. 5b). The specificity of the results was demonstrated by analyzing whether miR-29 overexpression affected the levels of other cytokines involved in Th17 or immunoregulatory responses. miR-29 was found not to affect expression of either IL-6 or TGF or IL-10 by qPCR (FIG. 5c), or of IL-10 and IL-6 by ELISA (FIGS. 5d and 5e) demonstrating a polarised role for miR-29 in regulation of IL-12p40 and IL-23p19, the subunits of IL-23.

miR-29 Decreases IL-17 Expression by Regulating IL-23, in DC+T Cell Co-Culture

The functional relevance of miR-29 repression of IL-12p40/IL-23p19 on the magnitude of Th17 responses was explored. DCs were transfected with miR-29 premiR or control before DC co-culture with CD4+ T cells. IL-17 production by T cells was then assessed at 72 h. This revealed significant reduction in IL-17 production from co-cultures where DCs over-expressed miR-29 (FIG. 7a and FIG. 10). Blocking antibodies to IL-23 and recombinant IL-23 (rIL-23) were then used to demonstrate this effect was indeed mediated by IL-23 (FIGS. 7b and 7c). The effect of DCs over-expressing miR-29 can be reversed and overcome by adding rIL-23 to these co-cultures (FIG. 7d). IFN expression from T cell co-cultures was shown to be unaffected by miR-29 expression in DCs (FIG. 7e). These results demonstrate miR-29 mediated control of IL-23 expression is functionally relevant in terms of magnitude of Th17 responses generated in its presence and absence.

Crohn's Patients DCs Expressing NOD2 Variants are Defective in miR-29 Expression and Down-Regulation of IL-12p40 Expression at the End of an Immune Response

Studies were undertaken to determine whether Crohn's patient DCs expressing variants of NOD2 associated with the disease exhibited defects in miR-29 up-regulation on either NOD2, or NOD2+ TLR, triggering. DCs were obtained from donors who had a previous histological diagnosis of Crohn's, and who were in remission and off immunotherapy at the time of sampling. FIGS. 8a and b show that these donors DCs fail to induce miR-29 on stimulation of NOD2, or NOD2+ TLR2, or NOD2+ TLR5 combined stimulation. The functional consequence for this absence in miR-29 expression was then investigated. It has already been established that NOD2 variants show defects in induction of IL-23 on ligand stimulation, such that there is absence of induction on NOD2 triggering alone and on dual NOD2+ TLR2 triggering the levels of IL-23 produced are reduced. These experiments reinforced this result at 24 h stimulation (FIG. 8c). Tests were then carried out to determine whether, despite the reduced levels of IL-12p40 initially induced in the presence of Crohn's associated NOD2 variants, the absence of miR-29 induction by NOD2 could affect return of IL-12p40 levels to background, following PRR activation. FIG. 8c demonstrates that Crohn's patient DCs expressing NOD2 variants display defects in reduction of IL-12p40 to basal levels at 72 h, post PRR triggering. Transfection of miR-29 into NOD2 FS1007insC DCs can reduce IL-12p40 expression (FIG. 8d), whereas blocking miR-29 effects in WT NOD2 DCs with an antimiR to miR-29 causes an increase in IL-12p40 expression (FIG. 8e). These experiments demonstrate Crohn's DCs expressing variant NOD2 fail to induce miR-29 following PRR stimulation, and that this leads to a reduced ability to return IL-12p40 to basal levels following combination PRR stimulation.

miR-29 Deficient Mice Develop Exacerbated Intestinal Inflammation Associated with an Enhanced Th17 Transcriptional Signature in Colonic Tissue

As IL-23 has been shown to drive colitis in animal models investigations were undertaken to determine whether a lack of miR-29 lowers the threshold for development of intestinal inflammation in vivo. Unlike in human cells, it was found that the expression of miR-29 family members was not substantially regulated after stimulation with NOD2 or TLR2 ligands. This was evident with both in vitro-derived BMDCs and splenic CD11c⁺ cDCs isolated after NOD2/TLR2 triggering in vivo. Despite this lack of induction, expression of the miR29a-b1 target gene I112p40 was substantially enhanced at the transcriptional level in BMDCs lacking miR29a-b1 after 24 and 48 hours of stimulation, compared to WT controls, with this dysregulation leading to enhanced IL-12p40 protein production by miR29a-b1 deficient BMDCs at 72 hours post-stimulation. Experiments also established whether miR-29 was capable of repressing IL-12p40 in murine DCs. miR-29 or control was transfected into murine bone marrow derived DCs pre and post-NOD2+ TLR2 simulation and I112p40 mRNA abundance was determined. miR-29 effectively suppressed IL-12p40 expression from murine DCs to levels comparable with results obtained in human DCs. As miR-29 exhibited similar control over IL-12p40 expression in murine cells as in the human setting, the contributions of miR-29 in an experimental model of mucosal pathology was studied.

Mice deficient in the miR-29a cluster (miR-29a-miR-29b-1), (miR-29a-b1 KO mice) or wild-type littermate controls with intact miR-29 were used, and challenged mice with 1.5-2% DSS in drinking water for 1 week. Mice deficient in miR-29 showed an enhanced propensity to develop colitis (1.7 fold higher colitis incidence compared to WT mice, FIG. 11A), as well as exhibiting significantly enhanced pathological score and weight loss (FIG. 11B-C). The enhanced severity of colitis in miR-29 deficient mice was associated with a marked ‘Th17-type’ transcriptional signature in distal colonic tissue. This included elevated expression of the miR-29-target genes I123a and I112b (FIG. 11D-E), as well as mRNA encoding the key Th17 cytokines IL-17A and GM-CSF (FIG. 11F-G); both of which are associated with Th17-mediated immunopathology32,33. In addition the Th17 subset-determining transcription factor RORγt (Rorc) was substantially elevated in distal colonic tissue of colitic miR-29 deficient mice compared to DSS-treated WT mice (FIG. 11H), while Gata3 and Tbx21 were either lower, or not changed, in colitic miR-29 deficient vs WT animals. Levels of Foxp3 were only slightly enhanced. Importantly, the enhanced colitis in miR-29a-b1 deficient mice was not associated with a general increase in inflammatory mediators. Levels of I111b, Tnfa and I16 mRNA were enhanced to the same degree in colonic tissue of WT and miR-29 deficient mice (FIG. 11I), and a lack of miR-29 did not impact upon colonic expression of I110. In addition, miR-29a-b1 KO mice also demonstrated significantly increased IL-23p19 protein production from intestinal mucosal tissue explants over controls following DSS challenge (FIG. 11J). Colitic miR-29 KO mice also exhibited changes in their intestinal mucosa in the expression of genes that we found modulated by miR-29 mimic in human DCs, including Cxc19, Cxc110, Clec7a, Cxc111, IL1f9 and Ifitm1, showing further parallels in miR-29 targeting between this murine model and human DCs. These results therefore demonstrate that a lack of miR-29 results in a dysregulation of IL-12 and IL-23 expression after PRR triggering in murine dendritic cells and enhanced expression of these cytokines in the colon during mucosal challenge, resulting in enhanced colonic inflammation, associated with a marked Th17-type transcriptional signature.

Crohn's Patients DCs Expressing NOD2 Variants are Defective in miR-29 Expression and Demonstrate Increased IL-12p40 Release on Exposure to Adherent Invasive E. coli

Whether Crohn's patient DCs expressing variants of NOD2 associated with the disease exhibited defects in miR-29 up-regulation on either NOD2, or NOD2+ TLR, triggering was investigated. DCs were obtained from donors who had previous histological diagnosis of Crohn's, and who were in remission and off immunotherapy for more than 6 months at the time of sampling. Donors either homozygous for 1007fsinsC NOD2 expression or compound heterozygous for any of the Crohn's associated NOD2 polymorphisms fail to induce miR-29 on stimulation of NOD2, or NOD2+ TLR2, or NOD2+ TLR5 combined stimulation (FIGS. 12A and 2B). In contrast these Crohn's donor DCs induced miR-155 to comparable levels to healthy wild type NOD2 expressing DCs following PRR triggering (FIG. 12C). Restoring miR-29 expression in Crohn's donor DCs expressing associated NOD2 polymorphisms effectively down-regulated IL-12p40 (FIG. 12D) as previously observed in healthy donors. Whether the loss of miR-29 induction by CD DCs expressing NOD2 polymorphisms might contribute to dysregulated IL-12p40 release from these cells on exposure to intestinal bacteria was explored. Healthy or CD DCs with AIEC were challenged and release of IL-12p40 was measured at day 3, day 5 and day 7 post exposure. A significantly enhanced release of IL-12p40 in CD donor cells at days 5 and 7 post challenge was observed (FIG. 12E). Whether introduction of miR-29 mimic into CD donor DCs could also reduce levels of IL-12p40 release after bacterial exposure was assessed and again an efficient reduction of IL-2p40 after transfection of miR-29 mimic was found, but not premiR control, into either healthy donor or CD donor cells exposed to AIEC (FIG. 12F). These observations demonstrate Crohn's donor DCs show defects in induction of miR-29 that correlate with enhanced IL-12p40 release following DC exposure to AIEC, suggesting that this may contribute to increased IL-23 levels observed in the intestinal mucosa during this disease.

### Discussion

The data presented herein demonstrates that NOD2 can control miRNA expression and that it induces the miR-29 family in DCs. This induction requires the key NOD2 signal transducer RIPK-2 and can be augmented by co-triggering NOD2 with either TLR2 or TLR5. A new series of targets for miR-29 were identified, namely: IL-12p40, ATF3 and SMAD3, whose down-regulation by miR-29 leads to down-regulation of IL-23 release from DCs. NOD2 and TLRs are key regulators of IL-23 production by cells of the innate immune system such as DCs and macrophages, so induction of miR-29 controlled by NOD2 represents a key intrinsic homeostatic mechanism to switch off this critical driver of Th17 responses. IL-23 can also be induced in DCs by endogenous signals such as prostaglandin E227 and stimulation via CD40L11, demonstrating a potential role for T cells in reinforcing the IL-23 response.

These studies demonstrate that Crohn's patient DCs homozygous or compound heterozygous for NOD2 variants associated with the disease failed to induce miR-29 to any significant level on NOD2 or NOD2+ TLR triggering. This may be used as a diagnostic tool and/or a therapeutic target for Crohn's disease.

1. A method of determining the Crohn's disease status of a subject comprising the steps of:
(c) determining the level of miR-29 in a sample from said subject; and
(d) comparing the level of miR-29 determined in step (a) with one or more reference values.

2. The method of claim 2 wherein the sample material comprises peripheral blood cells or myeloid derived cells.

3. The method of claim 1 or 2 wherein the level of miR-29 in the sample is determined pre and/or post pattern recognition receptor triggering of cells in the sample.

4. The method of any preceding claim wherein the reference value, to which the determined levels of miR-29 are compared, is the level of the miR-29 observed in one or more subjects that do not have any detectable Crohn's disease or any clinical symptoms of Crohn's disease, and have so called “normal values” of the biomarker miR-29.

5. The method of any preceding claim wherein if no increase in miR-29 levels is observed in a sample in response to PRR triggering, compared to the level in a normal sample, then this is diagnostic of Crohn's disease.

6. A kit for use in determining the Crohn's disease status of a subject comprising at least one agent for determining the level of miR-29 in a sample provided by the subject.

7. The kit of claim 6 wherein the agent is an antibody or a nucleic acid probe or one or more primers for use in a PCR reaction.

8. The kit of claim 6 or 7 further comprising instructions for suitable operational parameters in the form of a label or separate insert.

9. The kit of any of claims 6 to 8 further comprising one or more miRNA samples, to be used as standard(s) for calibration and comparison.

10. Use of the determination of the level of miR-29 in a sample from an individual as a means of assessing the Crohn's disease status of the individual.

11. A method of treating an inflammatory condition, and Crohn's disease in particular, in a subject comprising administering to the subject an agent capable of modulating the level of miR-29 in a cell.

12. The method of claim 11 wherein miR-29 level is modulated to increase the level of miR-29.

13. The method of claim 11 or 12 wherein level of miR-29 is increased by administering miR-29 to the cells.

14. The method of claim 11 or 12 wherein miR-29 levels are increased by preventing the inhibition of the natural expression of miR-29 in cells.

15. An agent for increasing miR-29 levels in a cell, or a population of cells, for use in the treatment of an inflammatory condition.

16. The use of an agent that increases the miR-29 levels in a cell in the preparation of a medicament for the treatment of an inflammatory condition.

17. The use of miR-29 as a therapeutic agent for the treatment of an inflammatory disease.

18. The use of claim 15, 16 or 17 wherein the inflammatory condition is Crohn's disease.

19. A method of identifying compounds for treating Crohn's disease comprising screening for one or more compounds that increase the level of miR-29 in vitro and/or in vivo.

20. A compound identified by the method of claim 19.


SEQUENCE LISTING (14 sequences)

SEQ ID NO: 1 - RNA, 64 nt, Homo sapiens
augacugauuucuuuugguguucagagucaauauaauuuucuagcaccaucugaaaucgguuau

SEQ ID NO: 2 - RNA, 81 nt, Homo sapiens
cuucaggaagcugguuucauauggugguuuagauuuaaauagugauugucuagcaccauuugaaaucaguguucuuggggg

SEQ ID NO: 3 - RNA, 81 nt, Homo sapiens
cuucuggaagcugguuucacaugguggcuuagauuuuuccaucuuuguaucuagcaccauuugaaaucaguguuuuaggag

SEQ ID NO: 4 - RNA, 88 nt, Homo sapiens
aucucuuacacaggcugaccgauuucuccugguguucagagucuguuuuugucuagcaccauuugaaaucgguuaugauguaggggga

SEQ ID NO: 5 - DNA, 24 bp, Artificial Sequence
  synthetic primer
gaattccttcacatcactttccag

SEQ ID NO: 6 - DNA, 23 bp, Artificial Sequence
  synthetic primer
gtcaacttgaggtgcccaacatt

SEQ ID NO: 7 - DNA, 18 bp, Artificial Sequence
  synthetic primer
cccagctcctccctcttc

SEQ ID NO: 8 - DNA, 21 bp, Artificial Sequence
  synthetic primer
aagtctgtaatgtaaagccac

SEQ ID NO: 9 - DNA, 20 bp, Artificial Sequence
  synthetic primer
ctgagcctttgttgatgagc

SEQ ID NO: 10 - DNA, 20 bp, Artificial Sequence
  synthetic primer
tcttcaaccacatccccatt

SEQ ID NO: 11 - DNA, 42 bp, Artificial Sequence
  synthetic primer
aaacgagctcgctagtaggttctgatccaggatgaaaatttg

SEQ ID NO: 12 - DNA, 48 bp, Artificial Sequence
  synthetic primer
gcaggtcgactctagtgattacaaagaagagtttttattagttcagcc

SEQ ID NO: 13 - DNA, 33 bp, Artificial Sequence
  synthetic primer
aaacgagctcgctagggcagcagctcaaggatg

SEQ ID NO: 14 - DNA, 41 bp, Artificial Sequence
  synthetic primer
gcaggtcgactctagagccacaaaaataagactttattgaa